(19) United States
(12) Patent Application Publication
(10) Pub. No.: US 2024/0058266 A1
(43) Pub. Date: Feb. 22, 2024

(54) Topical Composition of Pirfenidone

(71) Applicant: Cipla Limited, Mumbai (IN)

(72) Inventors: Trupti CHITRE, Mumbai (IN); Vinayak MARATHE, Mumbai (IN)

(21) Appl. No.: 18/267,577

(22) PCT Filed: Dec. 16, 2021
(86) PCT No.: PCT/IN2021/051180

§ 371 Date: Jun. 15, 2023

(30) Foreign Application Priority Data
Dec. 16, 2020 (IN) 202021054667

(51) Int. Cl. A61K 9/06; A61K 31/4418; A61K 47/18; A61K 47/20; A61K 47/14; A61K 47/10; A61K 47/32

(52) U.S. Cl. CPC A61K 9/06; A61K 31/4418; A61K 47/18; A61K 47/20; A61K 47/14; A61K 47/10; A61K 47/32

(57) ABSTRACT

The present invention relates to a topical composition comprising pirfenidone and process to prepare the same. The present invention also relates to use of the topical composition comprising pirfenidone in the treatment of skin disorders of the dermis of fibrous or inflammatory origin.

### FIELD OF INVENTION

The present invention relates to a topical composition comprising pirfenidone and process to prepare the same. The present invention also relates to use of the topical composition comprising pirfenidone in the treatment of skin disorders of the dermis of fibrous or inflammatory origin.

### BACKGROUND OF INVENTION

Pirfenidone is chemically known as -methyl-1-phenyl-2-1(H)-pyridone and is synthetic molecule with a molecular weight of 185.23 daltons. Pirfenidone is manufactured and marketed as a broad-spectrum anti-fibrotic drug. Pirfenidone has anti-fibrotic properties via: decreased TNF-.alpha. expression, decreased PDGF expression, and decreased collagen expression.

One important use of pirfenidone is known to be providing therapeutic benefits to patients by improving the appearance of the skin by diminishing abnormal scars, imperfections caused by surgical wounds, burns, accidents, sun, pregnancy, chemical agents, stains due to melasma, scarring, stretch marks, and wrinkles. Pirfenidone demonstrates a pharmacologic ability to prevent or remove excessive scar tissue found in fibrosis associated with injured skin. Published and unpublished basic and clinical research suggests that pirfenidone may safely slow or inhibit the progressive enlargement of fibrotic lesions, remove pre-existing fibrotic lesions, and prevent formation of new fibrotic lesions following tissue injuries.

WO00/16775 describes the use of pirfenidone for the treatment and prevention of skin lesions, especially fibrotic lesions, such as fibrotic damaged tissue, contact with infectious warts, contact dermatitis, burns and scars.

Topically applied medications are of particular advantage in skin disorders of the inflammatory and fibrotic origin as they can act locally in sufficient concentrations without the adverse effects encountered by systemic administration. Topical administration of pirfenidone for skin disorders of the inflammatory and fibrotic origin would be of particular advantage, given the increase in liver enzymes due to systemic administration of pirfenidone. Oral pirfenidone is known to cause an increase in liver enzymes and patient needs to monitor the liver profile while on oral pirfenidone. Further, gastrointestinal effects like nausea, diarrhea, dyspepsia, vomiting, gastro-esophageal reflux disease, and abdominal pain were prevalent on patients with oral pirfenidone.

There is a need in the art for topical pirfenidone compositions for treatment of skin disorders of the inflammatory and fibrotic origin, which can stay on the affected site for a longer period of time and which are easy to manufacture. The present invention provides a topical composition of pirfenidone for treatment of skin disorders of the inflammatory and fibrotic origin. The present invention also provides a topical composition of pirfenidone comprises atleast one thickening agent comprising acrylamide/sodium acryloyldimethyltaurate copolymer. The present invention further provides a topical composition of pirfenidone comprises atleast one thickening agent comprising polyacrylate crosspolymer-6 which has sufficiently high and consistently good viscosity and is easy to manufacture.

### OBJECT OF THE INVENTION

An object of the present invention is to provide a topical composition comprising pirfenidone and process to prepare the same.

Another object of the present invention is to provide a topical composition comprising pirfenidone, atleast one solubilizing agent, atleast one thickening agent comprising acrylamide/sodium acryloyldimethyltaurate copolymer and water. In yet another aspect, the composition further comprises a preservative. In one more aspect, the composition further comprises a surfactant.

One more object of the present invention is to provide a topical composition comprising pirfenidone, atleast one solubilizing agent, atleast one thickening agent comprising polyacrylate crosspolymer-6 and water. In one aspect, the composition further comprises a preservative. In a further aspect, the composition further comprises a surfactant.

Yet another object of the present invention is a method of treating skin disorders of the dermis of fibrous or inflammatory origin by the topical compositions provided herein.

### SUMMARY OF THE INVENTION

The present invention relates to a topical composition comprising pirfenidone and process to prepare the same. The present invention also relates to use of the topical composition comprising pirfenidone in the treatment of skin disorders of the dermis of fibrous or inflammatory origin.

In one embodiment, the topical composition comprises pirfenidone and pharmaceutically acceptable excipients.

In another embodiment, the topical composition comprises pirfenidone, atleast one solubilizing agent, atleast one thickening agent comprising acrylamide/sodium acryloyldimethyltaurate copolymer and water. In yet another embodiment, the composition further comprises a preservative. In one more embodiment, the composition further comprises a surfactant.

In a further embodiment, the topical composition comprises pirfenidone, atleast one solubilizing agent, atleast one thickening agent comprising polyacrylate crosspolymer-6 and water. In yet another embodiment, the composition further comprises a preservative. In one more embodiment, the composition further comprises a surfactant.

One more embodiment is a topical composition comprising about 5% w/w to about 15% w/w pirfenidone. In another embodiment, the topical composition comprises about 8% w/w pirfenidone.

In one embodiment, the topical composition comprises a solubilizing agent comprising propylene glycol, ethanol, diethylene glycol monoethyl ether, diethyl sebacate, light mineral oil, dimethyl isosorbide, isopropyl myristate, polyethylene glycol, hexylene glycol, and benzyl alcohol. In yet another embodiment, the solubilizing agent is present in an amount of about 5% w/w to about 40% w/w.

In one embodiment, the topical composition comprises a thickening agent comprising acrylamide/sodium acryloyldimethyltaurate copolymer in an amount of about 1% w/w to about 10% w/w. In a further embodiment, the topical composition comprises a thickening agent comprising acrylamide/sodium acryloyldimethyltaurate copolymer in an amount of about 1% w/w to about 4% w/w.

In another embodiment, the topical composition comprises a thickening agent comprising polyacrylate crosspolymer-6 in an amount of about 1% w/w to about 10% w/w. In one more embodiment, the topical composition comprises a thickening agent comprising polyacrylate crosspolymer-6 in an amount of about 1%w/w to about 3% w/w.

In one more embodiment, the topical composition comprising pirfenidone is a gel.

Yet another embodiment is a topical gel composition comprising about 8% w/w pirfenidone, about 4% acrylamide/sodium acryloyldimethyltaurate copolymer, about 30% w/w diethylene glycol monoethyl ether and water.

One embodiment is a topical gel composition comprising about 8% w/w pirfenidone, about 4% acrylamide/sodium acryloyldimethyltaurate copolymer, about 30% w/w diethylene glycol monoethyl ether, about 0.2% methylparaben and water.

A further embodiment is a topical gel composition comprising about 8% w/w pirfenidone, about 3% polyacrylate crosspolymer-6, about 30%w/w diethylene glycol monoethyl ether and water.

Another embodiment is a topical gel composition comprising about 8% w/w pirfenidone, about 3% polyacrylate crosspolymer-6, about 30%w/w diethylene glycol monoethyl ether, about 0.2% methylparaben and water.

One embodiment is a topical gel composition comprising about 8% w/w pirfenidone, about 3% polyacrylate crosspolymer-6, about 40% w/w diethylene glycol monoethyl ether and water.

Another embodiment is a topical gel composition comprising about 8% w/w pirfenidone, about 3% polyacrylate crosspolymer-6, about 40%w/w diethylene glycol monoethyl ether, about 0.2% methylparaben and water.

In one more embodiment, the topical composition provided herein is free of neutralizing agents.

In one embodiment, the pH of the topical composition may range from about 4 to about 9.

In one more embodiment, the pH of the topical composition may range from about 5 to about 7.

In another embodiment, the viscosity of the topical composition may range from about 0.5 poise to about 9 poise.

In one more embodiment, the topical composition provided herein has a long shelf life, i.e., it is stable during long term storage. The pharmaceutical composition or solution may contain greater than about 80%, such as greater than about 85%, greater than about 90%, greater than about 95% or greater than about 98% of the initial amount of pirfenidone after being stored for 3 or 6 months or 1, 2 or 3 years at 25° C.

Another embodiment is a method of treating skin disorders of the dermis of fibrous or inflammatory origin. In one embodiment, the topical composition comprising pirfenidone is applied atleast once daily. In yet another embodiment, the topical composition comprising pirfenidone is applied twice a day. In one more embodiment, the topical composition comprising pirfenidone is applied three times a day. In an embodiment, the topical composition comprising pirfenidone is applied at nighttime.

Other objects, features and advantages of the present invention will be apparent to those of ordinary skill in the art in view of the following detailed description of the invention and accompanying drawings.

### DETAILED DESCRIPTION OF THE INVENTION

The present invention relates to a topical composition comprising pirfenidone and process to prepare the same. The present invention also relates to use of the topical composition comprising pirfenidone in the treatment of skin disorders of the dermis of fibrous or inflammatory origin.

Pirfenidone is a drug that has been applied in the restoration of tissues with lesions with fibrosis and the prevention of fibrotic lesions. Pirfenidone is chemically identified as 5-methyl-1-phenyl-2-1(H)-pyridone and has the following chemical structure:

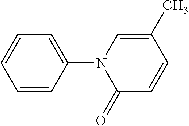

The topical composition of the present invention contains pirfenidone in amount of about 5% w/w to about 15% w/w. Preferably, the topical composition contains about 8% w/w pirfenidone.

The topical compositions comprising pirfenidone may further contain pharmaceutically acceptable excipients.

Pharmaceutically acceptable excipient include, but are not limited to, solubilizing agents, thickening agents, preservatives, and surfactants.

Solubilizing agents are of particular importance so as to solubilize the pirfenidone and maintain the pirfenidone in dissolved form throughout the shelf life. Solubilizing agents include, but are not limited to, propylene glycol, ethanol, diethylene glycol monoethyl ether, diethyl sebacate, light mineral oil, dimethyl isosorbide, isopropyl myristate, polyethylene glycol, hexylene glycol, and benzyl alcohol. The solubilizing agent can be present in an amount of about 5%w/w to about 40% w/w.

Thickening agents impart viscosity to the topical composition in sufficient amounts to improve the residence time of the topical composition when applied on the skin surface. The topical composition of the present invention comprises acrylamide/sodium acryloyldimethyltaurate copolymer as the thickening agent. Alternatively, the topical composition of the present invention may comprise polyacrylate crosspolymer-6 as the thickening agent. It has been observed that topical gel composition using acrylamide/sodium acryloyldimethyltaurate copolymer or polyacrylate crosspolymer-6 as thickening agent imparts sufficiently high and consistent viscosity than other thickening agents known in the art. Further, topical gel composition prepared with acrylamide/sodium acryloyldimethyltaurate copolymer or polyacrylate crosspolymer-6 as thickening agent eliminated the process of adding a neutralizing agent in the topical gel composition. Thus, addition of acrylamide/sodium acryloyldimethyltaurate copolymer or polyacrylate crosspolymer-6 as thickening agent provided a better composition and eliminated the need of additional excipients, and in addition provided an easier and quicker manufacturing process. Acrylamide/sodium acryloyldimethyltaurate copolymer is available in the brand name “Sepineo P600®”. Polyacrylate crosspolymer-6 is available in the brand name “Sepineo P.H.D 100”. In one embodiment, the topical composition comprises a thickening agent comprising acrylamide/sodium acryloyldimethyltaurate copolymer in an amount of about 1%w/w to about 10% w/w. In a further embodiment, the topical composition comprises a thickening agent comprising acrylamide/sodium acryloyldimethyltaurate copolymer in an amount of about 1%w/w to about 4% w/w. In another embodiment, the topical composition comprises a thickening agent comprising polyacrylate crosspolymer-6 in an amount of about 1% w/w to about 10% w/w. In one more embodiment, the topical composition comprises a thickening agent comprising polyacrylate crosspolymer-6 in an amount of about 1% w/w to about 3% w/w.

Preservatives may include, but are not limited to, parabens such as methyl paraben, propyl paraben, quaternary ammonium compounds, such as benzalkonium chloride, benzethonium chloride, cetrimide, dequalinium chloride, and cetylpyridinium chloride; alcoholic agents, such as, chlorobutanol; antibacterial esters, such as esters of parahydroxybenzoic acid.

Surfactants may include, but not limited to polysorbates such as polysorbate 20, polysorbate 40, polysorbate 60 or polysorbate 80, monoglyceride esters of C-22 fatty acids such as glyceryl monocaprylate, glyceryl monocaprate, glyceryl monostearate, glyceryl monobehenate, diglyceride esters of C-22 fatty acids such as glyceryl dilaurate, mono- and diglyceride esters of C-22 fatty acids such as caprylic/capric mono- and diglycerides or glyceryl mono- and diricinoleate, propylene glycol esters of C-22 fatty esters such as propylene glycol monocaprylate or propylene glycol monolaurate, dialkylene glycol monoalkyl ethers such as diethylene glycol monoethyl ether, polyglyceryl C-22 fatty acid esters such as polyglyceryl-3-diisostearate, polyethylene glycol esters of a triglyceride/vegetable oil containing 4-8 mole of ethylene oxide groups/mole of glyceride such as PEG-6 corn oil, PEG-6 almond oil, PEG-6 apricot kernel oil, PEG-6 olive oil, PEG-6 peanut oil, PEG-6 palm kernel oil or hydrogenated palm kernel oil, PEG-6 triolein or PEG-8 corn oil, polyethylene glycol ester of a vegetable oil containing at least 20 mole of ethylene oxide groups/mole of glyceride, such esters being selected from the group consisting of polyoxyethylene castor oil derivatives, e.g. PEG 20, 30, 35, 38, 40, 50 or 60 castor oil or PEG 20, 25, 30, 40, 45, 50, 60 or 80 hydrogenated castor oil, PEG 20 or 60 corn glycerides, PEG 20 or 60 almond glycerides or PEG 40 palm kernel oil, sodium laurate sulfate, a sucrose ester, e.g. sucrose stearate, sucrose distearate, sucrose cocoate or sucrose monolaurate, and polyglyceryl esters.

The topical composition of the present invention may be free of neutralizing agents, including, but not limited to triethanolamine (TEA), sodium bicarbonate, sodium hydroxide and potassium hydroxide. The topical composition of the present invention may have a pH ranging from about 4 to about 9, preferably about 5 to about 7. The viscosity of the topical composition of the present invention may range from about 0.5 poise to about 9 poise.

One embodiment is a topical composition comprising pirfenidone, atleast one solubilizing agent, atleast one thickening agent comprising acrylamide/sodium acryloyldimethyltaurate copolymer and water. In one embodiment, the composition further comprises a preservative. In yet another embodiment, the composition further comprises a surfactant. In one more embodiment, the topical composition is a gel.

Another embodiment is a topical composition comprising about 5% w/w to about 15% w/w pirfenidone, about 5% w/w to about 40% w/w solubilizing agent, about 1% w/w to about 10% w/w acrylamide/sodium acryloyldimethyltaurate copolymer as thickening agent, and water. The topical composition provided herein may optionally contain a preservative. In one embodiment, the topical composition is a gel.

A further embodiment is a topical gel composition comprising about 8%w/w pirfenidone, about 4% acrylamide/sodium acryloyldimethyltaurate copolymer, about 30% w/w diethylene glycol monoethyl ether and water. The topical gel composition provided herein may optionally contain about 0.2% w/w methylparaben as preservative.

One embodiment is a topical composition comprising pirfenidone, atleast one solubilizing agent, atleast one thickening agent comprising polyacrylate crosspolymer-6 and water. In one embodiment, the composition further comprises a preservative. In yet another embodiment, the composition further comprises a surfactant. In one more embodiment, the topical composition is a gel.

Another embodiment is a topical composition comprising about 5% w/w to about 15% w/w pirfenidone, about 5% w/w to about 40% w/w solubilizing agent, about 1% w/w to about 10% w/w polyacrylate crosspolymer-6 as thickening agent, and water. The topical composition provided herein may optionally contain a preservative. In one embodiment, the topical composition is a gel.

A further embodiment is a topical gel composition comprising about 8% w/w pirfenidone, about 3% polyacrylate crosspolymer-6, about 30% w/w diethylene glycol monoethyl ether and water. The topical gel composition provided herein may optionally contain about 0.2% w/w methylparaben as preservative

One embodiment is a topical gel composition comprising about 8% w/w pirfenidone, about 3% polyacrylate crosspolymer-6, about 40%w/w diethylene glycol monoethyl ether and water.

Another embodiment is a topical gel composition comprising about 8% w/w pirfenidone, about 3% polyacrylate crosspolymer-6, about 40%w/w diethylene glycol monoethyl ether, about 0.2% methylparaben and water.

The topical composition provided herein has a long shelf life, i.e., it is stable during long term storage. The pharmaceutical composition or solution may contain greater than about 80%, such as greater than about 85%, greater than about 90%, greater than about 95% or greater than about 98% of the initial amount of pirfenidone after being stored for 3 or 6 months or 1, 2 or 3 years at 25° C.

The topical composition of the present invention can be prepared as per the following process:
(i) Preparing a slurry of thickening agent (acrylamide/sodium acryloyldimethyltaurate copolymer or polyacrylate crosspolymer-6) in purified water.
(ii) Dissolving preservative in the solubilizing agent
(iii) Adding pirfenidone to step (ii) and stir to dissolve pirfenidone completely
(iv) Adding solution of step (iii) to step (i)
(v) Mixing bulk of step (iv) in a homogenizer to form uniform homogeneous gel

The present invention also relates to use of the topical composition comprising pirfenidone in the treatment of skin disorders of the dermis of fibrous or inflammatory origin.

“Skin disorders of the dermis of fibrous or inflammatory origin” mean improves the appearance of the skin by diminishing abnormal scars, imperfections caused by surgical wounds, burns, accidents, sun, pregnancy, chemical agents, stains due to melasma, scarring, stretch marks, and wrinkles.

In one embodiment, the topical composition comprising pirfenidone is applied atleast once daily. In yet another embodiment, the topical composition comprising pirfenidone is applied twice a day. In one more embodiment, the topical composition comprising pirfenidone is applied three times a day. In an embodiment, the topical composition comprising pirfenidone is applied at nighttime.

The present invention will now be explained with reference to the following non-limiting examples.

### Example 1

| Sr. No | Name of Ingredients | Function | Qty. (% w/w) |
|---|---|---|---|
| 1 | Pirfenidone | Active | 8.0 |
| 2 | Diethylene glycol | Solubilizing | 30.0 |
| | monoethyl ether | agent | |
| 3 | Methylparaben | Preservative | 0.2 |
| 4 | Acrylamide/sodium | Thickening | 4.0 |
| | acryloyldimethyltaurate | agent | |
| | copolymer, isohexadecane, | | |
| | polysorbate 80 | | |
| | (Sepineo P 600 ®) | | |
| 5 | Purified water | Vehicle | Q.S. to 100% |

Manufacturing Process:

Step 1: Preparation of slurry of Acrylamide/sodium acryloyldimethyltaurate copolymer (Sepineo P 600®))

Add Sepineo P 600® in purified water under stirring at room temperature and stir for 30 minutes to form lump free slurry.

Step 2: Preparation of Drug Phase (Pirfenidone):

Heat Diethylene glycol monoethyl ether at 50±5° C.

Add and dissolve methylparaben under stirring to form clear solution.

Add and dissolve Pirfenidone under stirring to get clear solution of drug phase.

Step 3: Drug Phase Addition:

Add drug phase of Step 2 to Sepineo P 600 slurry of Step 1 under homogenization. After complete addition of drug phase, homogenize bulk for 30 min.

Step 4: Mixing:

After complete homogenization mix bulk for 30 minutes

### Example 2

| Sr. No | Name of Ingredients | Function | Qty. (% w/w) |
|---|---|---|---|
| 1 | Pirfenidone | Active | 8.0 |
| 2 | Diethylene glycol | Solubilizing | 30.0 |
| | monoethyl ether | agent | |
| 3 | Methylparaben | Preservative | 0.2 |
| 4 | Polyacrylate | Thickening agent | 3.0 |
| | crosspolymer-6 | | |
| | (Sepineo P.H.D 100) | | |
| 5 | Purified water | Vehicle | Q.S. to 100% |

Manufacturing Process:

Step1: Preparation of Slurry of Polyacrylate Crosspolymer-6 (Sepineo P.H.D 100®)

Add Sepineo P.H.D 100® in purified water under stirring at room temperature and stir for 30 minutes to form lump free slurry.

Step 2: Preparation of Drug Phase (Pirfenidone):

Heat Diethylene glycol monoethyl ether at 50±5° C.

Add and dissolve methylparaben under stirring to form clear solution.

Add and dissolve Pirfenidone under stirring to get clear solution of drug phase.

Step 3: Drug Phase Addition:

Add drug phase of Step 2 to Sepineo P.H.D 100 slurry of Step 1 under homogenization. After complete addition of drug phase, homogenize bulk for 30 min.

Step 4: Mixing:

After complete homogenization mix bulk for 30 minutes

### Example 3

The topical gel compositions with acrylamide/sodium acryloyldimethyltaurate copolymer or polyacrylate crosspolymer-6 as thickening agent was compared with topical gel compositions prepared using xanthan gum as thickening agent.

| | | Compo- | Compo- | Compo- |
|---|---|---|---|---|
| | | sition 1 | sition 2 | sition 3 |
| | | Sepineo | Sepineo | Xanthan |
| Pirfenidone gel 8% | | P600 | P.H.D 100 | gum |
| Sr. No | Ingredients | (% w/w) | (% w/w) | (% w/w) |
| 1 | Pirfenidone | 8.0 | 8.0 | 8.0 |
| 2 | Diethylene glycol | 30.0 | 30.0 | 30.0 |
| | monoethyl ether | | | |
| | (Transcutol P) | | | |
| 3 | Methylparaben | 0.2 | 0.2 | 0.2 |
| 4 | Acrylamide/sodium | 4.0 | NA | NA |
| | acryloyldimethyl | | | |
| | taurate copolymer | | | |
| | (SEPINEO P 600) | | | |
| 5 | Polyacrylate | NA | 3.0 | NA |
| | crosspolymer-6 | | | |
| | (Sepineo P.H.D 100) | | | |
| 6 | Xanthan gum | NA | NA | 2.85 |
| 7 | Purified water | q.s | q.s | q.s |

Observations

| | | | | |
|---|---|---|---|---|
| 1 | Description | Opaque | Transparent | Opaque |
| | | gel | clear gel | gel |
| 2 | pH | 5.94 | 4.82 | 6.68 |
| 3 | Viscosity (poise) | 6.41 | 5.26 | 1.06 |

The rheological behavior of the three topical gel compositions were evaluated using Anton Paar MCR-302 rheometer.

A. Shear Thinning Behavior

A shear thinning behavior was evaluated for the three topical gel compositions and the observations are as given below:

| | Vis- | Vis- | Vis- | Vis- | Vis- |
|---|---|---|---|---|---|
| | cosity | cosity | cosity | cosity | cosity |
| | (P) at | (P) at | (P) at | (P) at | (P) at |
| | 0.01 | 0.1 | 1 | 10 | 100 |
| | shear | shear | shear | shear | shear |
| Composition | rate | rate | rate | rate | rate |
| Composition with | 79800 | 8930 | 1330 | 237 | 44.80 |
| acrylamide/sodium | | | | | |
| acryloyldimethyl | | | | | |
| taurate copolymer | | | | | |
| (SEPINEO P 600) | | | | | |
| Composition with | 14300 | 2410 | 418 | 93.1 | 25.50 |
| polyacrylate | | | | | |
| crosspolymer-6 | | | | | |
| (Sepineo P.H.D 100) | | | | | |
| Composition with | 86100 | 7980 | 522 | 65.3 | 8.99 |
| Xanthan Gum | | | | | |

It was observed that topical gel compositions using acrylamide/sodium acryloyldimethyl taurate copolymer and polyacrylate crosspolymer-6 demonstrated the highest viscosity over the range of shear rates as compared to that with xanthan gum as thickening agent. It was also observed that topical gel compositions with acrylamide/sodium acryloyldimethyl taurate copolymer and polyacrylate crosspolymer-6 were able to maintain a higher viscosity at high shear rates as compared to that with xanthan gum as thickening agent. Further, high shear viscosity drop was observed to a greater extent for topical gel compositions with xanthan gum as compared to topical gel composition with acrylamide/sodium acryloyldimethyl taurate copolymer and polyacrylate crosspolymer-6.

B. Measurement of Yield Stress Value and Linear Viscoelastic Range

The topical gel compositions were evaluated for rheology and yield stress value and linear viscoelastic range were measured. The result of the evaluation is as given in below table.

| | | LVE range (%) |
|---|---|---|
| | Yield stress | Linear visco- |
| Composition | value (Pa) | elastic range |
| Composition with | 78.8 | 2.74 |
| acrylamide/sodium | | |
| acryloyldimethyl taurate | | |
| copolymer (SEPINEO P 600) | | |
| Composition with | 15 | 6.61 |
| Polyacrylate crosspolymer- | | |
| 6 (Sepineo PHD) | | |
| Composition with Xanthan | due to noise in data | due to noise in data |
| Gum | no linear region | no linear region |
| | obtained for results | obtained for results |
| | calculation | calculation |

The above table demonstrates that the topical gel composition with acrylamide/sodium acryloyldimethyl taurate copolymer (Sepineo P600) and Polyacrylate crosspolymer-6 (Sepineo PHD) as thickening agents has a much better structural strength and elasticity The topical gel composition using xanthan gum does not appear to be structurally stable due to noise observed in the data.

### Example 4

| | | Qty./Unit |
|---|---|---|
| Sr. No | Ingredients | % w/w |
| 1 | Pirfenidone | 8 |
| 2 | Diethylene glycol monoethyl ether | 40 |
| | (Transcutol P) | |
| 3 | Methylparaben | 0.2 |
| 4 | Polyacrylate crosspolymer-6 | 3 |
| | (Sepineo P.H.D 100) | |
| 5 | Purified water | q.s 100%(48.8) |

Manufacturing Process:

Step1: Preparation of Slurry of Polyacrylate Crosspolymer-6 (Sepineo P.H.D 100®)

Add Sepineo P.H.D 100® in purified water under stirring at room temperature and stir for 30 minutes to form lump free slurry.

Step 2: Preparation of Drug Phase (Pirfenidone):

Heat Diethylene glycol monoethyl ether at 50±5° C.

Add and dissolve methylparaben under stirring to form clear solution.

Add and dissolve Pirfenidone under stirring to get clear solution of drug phase.

Step 3: Drug Phase Addition:

Add drug phase of Step 2 to slurry of Step 1 under homogenization. After complete addition of drug phase, homogenize bulk for 30 min.

Step 4: Mixing:

After complete homogenization mix bulk for 30 minutes

The above compositions were manufactured in triplicates and were analysed for physical stability (appearance) and chemical stability (assay of Pirfenidone and assay of preservative- Methylparaben). The below table depicts the stability data evaluated under different conditions and time periods.

| | | | | | | | Related | |
|---|---|---|---|---|---|---|---|---|
| | | | | | | | substances | |
| | | | | | | | | % |
| | | | | | | | % | Total |
| | | | Assay | Assay | | | Single | Un- |
| | | | of | of | | | Maximum | known |
| | | | Pirfen- | Methyl- | | Vis- | Unknown | Im- |
| Batch | | | idone | paraben | | cosity | Impurities | purities |
| no. | Condition | Description | % | % | pH | P | % | % |
| 1 | Initial | Transparent | 103.8 | 100.3 | 5.03 | 5.66 | <0.1 | <0.1 |
| | | gel | | | | | | |
| | 25/60- | Colourless | 100.8 | 99.4 | 4.48 | 5.49 | <0.1 | <0.1 |
| | 6 Months | to yellow | | | | | | |
| | | color hazy | | | | | | |
| | | gel | | | | | | |
| | 30/75- | Colourless | 100.2 | 98.7 | 4.35 | 5.42 | 0.1 | 0.1 |
| | 6 Months | to yellow | | | | | | |
| | | color hazy | | | | | | |
| | | gel | | | | | | |
| | 40/75- | Colourless | 99.7 | 99 | 4.04 | 4.5 | 0.3 | 0.5 |
| | 6 Months | to yellow | | | | | | |
| | | color hazy | | | | | | |
| | | gel | | | | | | |
| 2 | Initial | Transparent | 103.5 | 103.3 | 5.15 | 5.69 | <0.1 | <0.1 |
| | | gel | | | | | | |
| | 25/60- | Colourless | 100.8 | 101.2 | 4.36 | 5.53 | <0.1 | <0.1 |
| | 6 Months | to yellow | | | | | | |
| | | color hazy | | | | | | |
| | | gel | | | | | | |
| | 30/75- | Colourless | 100.8 | 101.2 | 4.18 | 5.35 | 0.1 | 0.1 |
| | 6 Months | to yellow | | | | | | |
| | | color hazy | | | | | | |
| | | gel | | | | | | |
| | 40/75- | Colourless | 99 | 99.6 | 3.95 | 4.39 | 0.3 | 0.5 |
| | 6 Months | to yellow | | | | | | |
| | | color hazy | | | | | | |
| | | gel | | | | | | |
| 3 | Initial | Transparent | 102.9 | 102.9 | 5.04 | 5.6 | <0.1 | <0.1 |
| | | gel | | | | | | |
| | 25/60- | Colourless | 100.7 | 101.5 | 4.29 | 5.61 | <0.1 | <0.1 |
| | 6 Months | to yellow | | | | | | |
| | | color hazy | | | | | | |
| | | gel | | | | | | |
| | 30/75- | Colourless | 101.1 | 100.9 | 4.23 | 5.36 | 0.1 | 0.1 |
| | 6 Months | to yellow | | | | | | |
| | | color hazy | | | | | | |
| | | gel | | | | | | |
| | 40/75- | Colourless | 99.4 | 100.7 | 4.03 | 4.66 | 0.2 | 0.4 |
| | 6 Months | to yellow | | | | | | |
| | | color hazy | | | | | | |
| | | gel | | | | | | |

Although the invention herein has been described with reference to particular embodiments, it is to be understood that these embodiments are merely illustrative of the principles and application of the present invention. It is therefore to be understood that numerous modifications may be made to the illustrative embodiments and that other arrangements may be devised without departing from the spirit and scope of the present invention as described.

1. A topical composition comprising pirfenidone, at least one thickening agent comprising polyacrylate crosspolymer-6, at least one solubilizing agent, optionally a preservative and water.

2. A topical composition comprising pirfenidone, at least one thickening agent comprising acrylamide/sodium acryloyldimethyltaurate copolymer, one solubilizing agent, optionally a preservative and water.

3. The topical composition according to claim 1, wherein the composition is a gel.

4. The topical composition according to claim 1, wherein the pirfenidone is present at a concentration of about 5% w/w to about 15% w/w.

5. The topical composition according to claim 1, wherein the pirfenidone is present at a concentration of about 8% w/w.

6. The topical composition according to claim 1, wherein the thickening agent is present at a concentration of about 1% w/w to about 10% w/w.

7. The topical composition according to claim 1, wherein the thickening agent is present at a concentration of about 1% w/w to about 3% w/w.

8. The topical composition according to claim 2, wherein the thickening agent is present at a concentration of about 1% w/w to about 4% w/w.

9. The topical composition according to claim 1, wherein the preservative is selected from methyl paraben, propyl paraben, benzalkonium chloride, benzethonium chloride, cetrimide, dequalinium chloride, cetylpyridinium chloride, chlorobutanol; and-or esters of parahydroxybenzoic acid.

10. The topical composition according to claim 1, wherein the solubilizing agent is selected from propylene glycol, ethanol, diethylene glycol monoethyl ether, diethyl sebacate, light mineral oil, dimethyl isosorbide, isopropyl myristate, polyethylene glycol, hexylene glycol, arid or benzyl alcohol.

11. The topical composition according to claim 1, wherein the solubilizing agent is present at a concentration of about 5% w/w to about 40% w/w.

12. The topical composition according to claim 1, wherein the solubilizing agent is present at a concentration of about 40% w/w.

13. A topical composition comprising about 8% w/w pirfenidone, about 3% polyacrylate crosspolymer-6, about 40% w/w diethylene glycol monoethyl ether, about 0.2% methylparaben and water.

14. A topical composition according to claim 1, wherein the composition is free of neutralizing agents.

15. A topical composition according to claim 1, wherein the composition has a pH of about 4 to about 9.

16. A topical composition according to claim 1, wherein the composition has a viscosity of about 0.5 poise to about 9 poise.

17. A topical composition according to claim 1, for use in treatment of skin disorders of the dermis of fibrous or inflammatory origin.

18. A topical composition according to claim 17, wherein the composition is applied at least once daily.

19. A topical composition according to claim 17, wherein the composition is applied twice a day.

20. A topical composition according to claim 17, wherein the composition is applied three times a day.

21. A topical composition according to claim 17, wherein the composition is applied at nighttime.
